(19) United States
(12) Patent Application Publication
(10) Pub. No.: US 2016/0213605 A1
(43) Pub. Date: Jul. 28, 2016

(54) A composition comprising an extract of combined herbs consisting of Acanthopanax koreanum Nakai and Crinum asiaticum var. japonicum showing preventing activity of baldness and stimulating activity of hair growth

(71) Applicant: SAM CHUN DANG PHARM. CO., LTD, Hwaseong-si, Gyeonggi-do (KR)

(72) Inventors: Guang Jin IM, Gyeonggi-do (KR); Gyo Cheol GU, Gyeonggi-do (KR); Seon Hwa JUNG, Gyeonggi-do (KR); Tae Hee LEE, Gyeonggi-do (KR); Jae Hyuk KWON, Gyeonggi-do (KR); Sun Hee LIM, Gyeonggi-do (KR); Jae Ho LEE, Gyeonggi-do (KR)

(21) Appl. No.: 14/915,898

(22) PCT Filed: Sep. 2, 2014
(86) PCT No.: PCT/KR2014/008175

§ 371 Date: Mar. 2, 2016

(30) Foreign Application Priority Data
Sep. 12, 2013 (KR) 10-2013-0109715

(51) Int. Cl. A61K 8/97; A61Q 7/00

(52) U.S. Cl. CPC A61K 8/97; A61Q 7/00; A61K 2800/80; A61K 2800/5922

(57) ABSTRACT

A composition comprising an extract of combined herbs Acanthopanax koreanum Nakai and Crinum asiaticum var. japonicum showing treatment activity of a hair baldness disorder and stimulating activity of hair growth. The combined extract showed potent hair-growth promoting activity and a synergistic effect compared to each herb, i.e. the sole treatment of Acanthopanax koreanum Nakai or Crinum asiaticum var. japonicum, through animal model experiments, such as, the growth rate test using C57BL/6 mouse, to confirm the effect of the combined extract, i.e. combined treatment of Acanthopanax koreanum Nakai and Crinum asiaticum var. japonicum. Therefore the extract can be useful in treating a hair baldness disorder.

### TECHNICAL FIELD

The present invention relates to a composition comprising an extract of combined herbs consisting of Acanthopanax koreanum Nakai and Crinum asiaticum var. japonicum showing preventing activity of baldness and stimulating activity of hair growth.

### BACKGROUND ART

Hair follicle, a very complex organ, consists of an inner root sheath (IRS), outer root sheath (ORS), hair shaft, hair matrix cell (Paus et al., J. Invest. Dermatol., 113, pp523-532, 1999), and dermal papilla cell, which plays an important role in hair growth among various kinds of the cells forming the hair follicle (Ferraris et al., Exp. Cell Res., 10, pp37-46, 1997).

Hair follicle develops through the interaction between the mesenchymal cell and epithelial cell during an embryogenesis. Normal hair growth consists of repeated three phases, i.e., anagen, catagen and telogen stages (Paus et al., N. Engl. J. Med., 341, pp491-497, 1999). Especially, human hair has the longer growth period than any other animal hair or the other part hairs of the human body (anagen, 2-5 years; catagen, several days-several weeks; telogen, three months). During the anagen stage, the cell consisting of hair follicle including the keratinocytic cell of matrix surrounding the dermal papilla, proliferates and grows hair. During the catagen stage, within the hair follicle, apoptosis, condensation of the dermal papilla, and the reconstitution of extracellular matrix serially occur (Paus et al., J. Invest. Dermatol., 113, pp523-532, 1999) to stop the cell division and slow the hair growth. The hair follicle at telogen stage falls out except the permanently lasting parts comprising the bulge region of hair follicle at the state of quiescence of growth to restart new anagen (Hardy M H., Trends Genet., 8, pp55-61, 1992).

Several drugs promoting hair growth approved by the Food and Drug Administration (FDA) have been developed, for example, ‘Minoxidil’ (Buhl et al., J. Invest. Dermatol., 92(3), pp315-320, 1989) and ‘Finasteride’ (Van Neste et al., Br. J. Dermatol., 143(4), pp804-810, 2000).

As an alternative approach, there have been attempts to develop new therapy using herb extract having hair growth stimulating effect: for example, an extract of Crinum asiaticum Linne var. japonicum Baker or the lycorine compound isolated therefrom disclosed in Korea Patent Registration No, 10-0979269; an extract of Acanthopanax koreanum Nakai disclosed in Korea Patent Registration Nos, 10-1151587 & 10-1151555, etc.

However, there have been still needed to develop new drugs or substances showing more potent activity without adverse action till now.

Crinum asiaticum Linne var. japonicum Baker belonged to Amaryllidaceae is distributed to Korea, tropical asia, Japan, North America. It has been reported to comprise various ingredients, for example, phenanthridine alkaloids, triterpene alcohols, and flavonoid (Min B S, et al., Cytotoxic alkaloids and a flavan from the bulbs of Crinum asiaticum var. japonicum. Chem Pharm Bull (Tokyo). 49(9):1217-1219, 2001) and to show various medicinal effect, for example, anti-viral activity of various alkaloids (Gabrielsen B, et al., Antiviral (RNA) activity of selected Amaryllidaceae isoquinoline constituents and synthesis of related substances. J Nat Prod. 55(11):1569-1581, 1992), anti-malarial activity (Likhitwitayawuid K, et al., Cytotoxic and antimalarial alkaloids from the bulbs of Crinum amabile. J Nat Prod. 56(8):1331-1338, 1993), cyto-toxicity activity (Abdel-Halim Oft et al., New crinine-type alkaloids with inhibitory effect on induction of inducible nitric oxide synthase from Crinum yemense. J Nat Prod. 67(7):1119-1124, 2004), etc.

Acanthopanax koreanum Nakai belonged to Araliaceae is distributed to only Cheju island in Korea. It has been reported to comprise various ingredients, for example, syringaresinol diglycoside (Kim Y H, et al., Studies on the constituents of Acanthopanax koreanum Nakai (1)., Kor. J. Pharmacogn, 16, pp. 151-154, 1985), acanthoside D, syringoside (Hahn D R, et al., A study on the chemical constituents of Acanthopanax koreanum and its pharmacobiological activities., Yakhak Hoeji, 29, pp. 357-361, 1985), eleutheroside B and E (Chung B S, et al., Studies on the constituents of Acanthopanax koreanum Nakai., Kor. J. Pharmacogn, 17, pp. 62-66, 1986), falcarindol, methyl n-hexacosanoate, and coniferin (Kim Y H, et al., Studies on the chemical constituents of Acanthopanax koreanum, Arch. Pharm. Res, 11, pp. 159-162, 1988), pimaradine diterpene (Kim Y H, et al., Pimaradine diterpenes from Acanthopanax koreanum. J. Nat. Prod, 51, pp. 1080-1082, 1988), sumogaside (Kim Y H, et al., Diterpene glycoside form Acanthopanax koreanum., Kor. J. Pharmacogn, 21, pp. 49-51, 1990), lupane glycosides (Choi H S, et al., Lupane glycosides from the leaves of Acanthopanax koreanum., Chem. Pharm. Bull, 56, pp. 1613-1616), etc and to show anti-rheumatic activity, ant-diabetic activity, hepato-protective activity etc (Perry L M, et al., Medicinal Plants of East and Southeast Asia. MIT Press, Cambrige, p. 41, 1980).

However, there has been not reported or disclosed about the hair growing activity of the extract of combined herbs consisting of Acanthopanax koreanum Nakai and Crinum asiaticum var. japonicum in any of above cited literatures, the disclosures of which are incorporated herein by reference.

Therefore, the inventors of the present invention evaluated the treating effect of the extract of combined herbs consisting of Acanthopanax koreanum Nakai and Crinum asiaticum var. japonicum on baldness disorder by observing the preventing activity of baldness disorder and stimulating activity of hair growth.

Accordingly, the inventors of the present invention carried out an animal model experiments, for example, the growth rate test using by C57BL/6 mouse, to confirm the effect of the combined herbs consisting of Acanthopanax koreanum Nakai and Crinum asiaticum var. japonicum, on hair growth, and finally completed the present invention by confirming the potent activity of hair growth.

These and other objects of the present invention will become apparent from the detailed disclosure of the present invention provided hereinafter.

### DISCLOSURE

### Technical Problem

The present invention relates to an external pharmaceutical composition comprising an extract of combined herbs consisting of Acanthopanax koreanum Nakai and Crinum asiaticum var. japonicum as active ingredients for preventing and treating hair baldness and stimulating activity of hair growth and the use thereof.

The present invention relates to an cosmetic composition comprising an extract of combined herbs consisting of Acanthopanax koreanum Nakai and Crinum asiaticum var. japonicum as active ingredients for preventing and improving hair baldness and stimulating activity of hair growth and the use thereof.

### Technical Solution

It is an object of the present invention to provide an external pharmaceutical composition comprising an extract of combined herbs consisting of Acanthopanax koreanum Nakai and Crinum asiaticum var. japonicum as active ingredients for preventing and treating hair baldness and stimulating activity of hair growth.

It is an object of the present invention to provide a cosmetic composition comprising an extract of combined herbs consisting of Acanthopanax koreanum Nakai and Crinum asiaticum var. japonicum as active ingredients for preventing and improving hair baldness and stimulating activity of hair growth.

The term “extract” disclosed herein includes polar solvent soluble extract, for example, the extract soluble in water, spirit, lower alkyl alcohol, or the mixture thereof, preferably, the mixture solvent with water and ethanol or spirit, more preferably from 10 to 100% ethanol or spirit, more and more preferably from 50 to 100% ethanol or spirit. The term “extract of combined herbs” disclosed herein comprises the extract of combined herbs consisting of dried Acanthopanax koreanum Nakai and Crinum asiaticum var. japonicum with mixed ratio of 1-10:10-1 (w/w), preferably, 1-5:5-1 (w/w), more preferably, 1-3:3-1 (w/w); or the combined extract of Acanthopanax koreanum Nakai and Crinum asiaticum var. japonicum with mixed ratio of 1-10:10-1 (w/w), preferably, 1-5:5-1 (w/w), more preferably, 1-3:3-1 (w/w).

The term “baldness disorder” disclosed herein comprises androgentic alopecia, alopecia seniles, alopecia areata and the like.

The present invention also provided a use of the an extract of combined herbs consisting of Acanthopanax koreanum Nakai and Crinum asiaticum var. japonicum for the preparation of therapeutic agent for the treatment and prevention of baldness disorder in mammal or human.

The present invention also provided a pharmaceutical composition comprising an extract of combined herbs consisting of Acanthopanax koreanum Nakai and Crinum asiaticum var. japonicum, and a pharmaceutically acceptable carrier thereof as an active ingredient for treating and preventing baldness disorder.

It is an object of the present invention to provide a method of treating or preventing baldness disorder in mammal or human in need thereof comprising administering to said mammal or human with an effective amount of an extract of combined herbs consisting of Acanthopanax koreanum Nakai and Crinum asiaticum var. japonicum, together with a pharmaceutically acceptable carrier thereof.

Hereinafter, the present invention is described in detail.

An inventive extract of the present invention can be prepared in detail by following procedures.

The inventive combined extract of the herbs consisting of Acanthopanax koreanum Nakai and Crinum asiaticum var. japonicum, can be prepared by the followings.

Dried Acanthopanax koreanum Nakai and Crinum asiaticum var. japonicum were cut and approximately 1 to 100-fold weight, preferably 5 to 20-fold weight of water, spirit, lower alkyl alcohol, or the mixture thereof, preferably, the mixture solvent with water and ethanol or spirit, more preferably from 10 to 100% ethanol or spirit, more and more preferably from 50 to 100% ethanol or spirit, were added and extracted at the temperature ranging from 40° C. to 160° C. preferably, 80° C. to 120° C. for the period ranging from 1 hour to 10 hours, preferably 2 hour to 6 hours by hot water-extraction, cold water-extraction, reflux extraction or ultra-sonication extraction, preferably, hot water-extraction or reflux extraction; the upper residue was filtered, concentrated and dried to obtain the powdered extract of each herb; the powdered extract was mixed with mixed ratio of 1-10:10-1 (w/w), preferably, 1-5:5-1 (w/w), more preferably, 1-3:3-1 (w/w) to obtain the inventive combined extract of the present invention.

In an alternative method, the inventive extract of combined herbs consisting of Acanthopanax koreanum Nakai and Crinum asiaticum var. japonicum, can be prepared by the followings.

Dried Acanthopanax koreanum Nakai and Crinum asiaticum var. japonicum were mixed with mixed ratio of 1-10:10-1 (w/w), preferably, 1-5:5-1 (w/w), more preferably, 1-3:3-1 (w/w), the mixture was cut and approximately 1 to 100-fold weight, preferably 5 to 20-fold weight of water, spirit, lower alkyl alcohol, or the mixture thereof, preferably, the mixture solvent with water and ethanol or spirit, more preferably from 10 to 100% ethanol or spirit, more and more preferably from 50 to 100% ethanol or spirit, were added and extracted at the temperature ranging from 40° C. to 160° C. preferably, 80° C. to 120° C. for the period ranging from 1 hour to 10 hours, preferably 2 hour to 6 hours by hot water-extraction, cold water-extraction, reflux extraction or ultra-sonication extraction, preferably, hot water-extraction or reflux extraction; the upper residue was filtered, concentrated and dried to obtain the inventive combined extract of the present invention.

It has been confirmed that the inventive combined extract showed potent hair-growth promoting activity and synergistic effect than each herb, i.e., the sole treatment of Acanthopanax koreanum Nakai and Crinum asiaticum var. japonicum, through the animal model experiments such as the growth rate test using by C57BL/6 mouse, to confirm the effect the inventive combined extract, i.e., combined treatment of Acanthopanax koreanum Nakai and Crinum asiaticum var. japonicum.

The present invention also provided a use of the an extract of combined herbs consisting of Acanthopanax koreanum Nakai and Crinum asiaticum var. japonicum prepared from the above-described method for the preparation of therapeutic agent for the treatment and prevention of baldness disorder in mammal or human.

The present invention also provided a pharmaceutical composition comprising the an extract of combined herbs consisting of Acanthopanax koreanum Nakai and Crinum asiaticum var. japonicum, prepared from the above-described method and a pharmaceutically acceptable carrier thereof as an active ingredient for treating and preventing baldness disorder.

It is an object of the present invention to provide a method of treating or preventing baldness disorder in mammal or human in need thereof comprising administering to said mammal or human with an effective amount of the an extract of combined herbs consisting of Acanthopanax koreanum Nakai and Crinum asiaticum var. japonicum, prepared from the above-described method together with a pharmaceutically acceptable carrier thereof.

The inventive composition for preventing baldness disorder and stimulating hair growth may comprise the above-described extract as 0.1-50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co., Easton Pa.).

Hereinafter, the following formulation methods and excipients are merely exemplary and in no way limit the invention.

The external pharmaceutical composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection.

Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

The external pharmaceutical composition containing present composition may be prepared in any form, such as a topical preparation, for example, cream, gel, patch, ointment, spray solution, liniment, cataplasma, lotion, gel, balm, paste, aerosol and the like; or injectable preparation (solution, suspension, emulsion). In particular, the composition of the present invention may be applied to topical regions such as skin or hair as a form of topical preparation, for example ointment, lotion, liniment, pasta, cataplasma and etc. which are not intended to limit thereto.

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract of the present composition varies depending on the condition and the weight of the subject, severity, drug form, route and period of administration, and may be chosen by those skilled in the art. However, in order to obtain desirable effects, it is generally recommended to administer at the amount ranging from 0.0001 to 11000 mg/kg, preferably 0.001 to 100 mg/kg by weight/day of the inventive extract or compounds of the present invention. The dose may be administered in single or divided into several times per day. In terms of composition, the amount of inventive extract should be present between 0.000001 to 50% by weight, preferably 0.0001 to 20% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The inventive composition for preventing baldness and stimulating hair growth may comprise the above-described extract as 0.01-10%, preferably 0.1-8%, more preferably 0.5-5% by weight based on the total weight of the composition.

The other components may be a mixture of the ingredients of a conventional cosmetic composition well known in the art.

Cosmetic formulations containing above composition may be prepared in any form such as hair tonic, hair conditioner, hair essence, hair lotion, hair nutrient lotion, hair shampoo, hair rinse, hair treatment, hair cream, hair nutrient cream, hair was, hair moisture cream, hair massage cream, hair aerosol, hair pack, hair nutrient pack, hair soap, hair cleaning foam, pomade, hair drying agent, hair care agent, hair dyeing agent, hair permanent wave agent, hair bleaching agent, hair gel, hair glaze, hair dressing pomade, hair lacquer, hair moisturizer, hair mousse, eyebrows nutrient, eyelash nutrient, hair spray, astringent, nutrient lotion, nutrient cream, massage cream, essence, pack, foundation, cleansing water, soap, treatment, beauty solution and the like.

The cosmetic composition of the present invention can comprises additional additives selected from the group consisting of water soluble vitamin, lipid soluble vitamin, peptide polymer, polysaccharide polymer, sphingolipid and sea-weed extract. Preferable water soluble vitamins are any one which can be mixed with cosmetic, however, various vitamin such as vitamin B₁, B₂, B₆, pyridoxine, pyridoxine HCl, vitamin B₁₂, pantothenic acid, nicotinic acid, nicotinamide, folic acid, vitamin C, vitamin H etc, the salt thereof such as thiamin HCl salt, ascorbic acid Na salt etc or their derivatives such as ascorbic acid-2-phosphonic acid Na salt, ascorbic acid-2-phosphonic acid Mg salt are preferable and those can be obtained by conventional method such as microbial conversion method, purification method from the microbial cultivates, enzymatic method or chemical synthetic method.

Preferable lipid soluble vitamins are any one which can be mixed with cosmetic, however, various vitamin such as vitamin A, D₂, D₃, E dl-tocopherol, d-tocopherol, l-tocopherol and their derivatives such as palmitic acid ascorbate, stearic acid ascorbate, dipalmitic acid ascorbate, acetic acid-dl-tocopherol, nicotinic acid dl-tocopherol vitamin E, dl-pantothenyl alcohol, D-pantothenyl alcohol, pantothenyl ethylether etc. including the lipid soluble vitamin used in examples of present invention are preferable and those can be obtained by conventional method such as microbial conversion method, purification method from the microbial cultivates, enzymatic method or chemical synthetic method.

Preferable peptide polymers are any one which can be mixed with cosmetic, however, collagen, hydrolysable collagen, gelatin, elastin, hydrolysable gelatin, keratin etc. including the peptide polymer used in examples of present invention are preferable. Preferable polysaccharide polymers are any one which can be mixed with cosmetic, however, hydroxy ethyl cellulose, xanthin gum, hyaluronic acid Na, chondroitin sulfate or their salt (Na salt etc) and the like are preferable. For example, chondroitin sulfate or the salt thereof etc can be used by being purified from mammal or fishes ordinarily.

Preferable sphingolipid are any one which can be mixed with cosmetic, however, ceramide, pit-sphingosin, sphingo-lipopolysaccharide and the like are preferable. Sphingo-lipid can be obtained by being purified from mammal, fish, shellfish, yeast or plant etc in conventional method.

Preferable seaweed extract is any one which can be mixed with cosmetic, however, the extract of brown algae, red algae, green algae and the like or the purified carrageenan, alginic acid, arginic acid Na, K isolated therefrom are preferable. Algae extract can be obtained by being purified from seaweed in conventional method.

The cosmetic composition of the present invention may combine with other ingredients used in conventional cosmetic composition, if necessary, together with above described essential ingredient.

Preferable above described other ingredients may comprise oil ingredient, humectants, emollients, surfactants, organic or inorganic dye, organic powder, ultraviolet ray absorbing agent, preservatives, antiseptics, antioxidants, plant extract, pH controller, alcohol, pigments, perfumes, refrigerants, blood circulator, antihidrotic, distilled water and etc.

Preferable oil ingredients may comprise ester oil, hydrocarbon oil, silicone oil, fluoride oil, animal oil, plant oil and so on.

Preferable ester oil described above may comprise glyceryl tri-2-ethyl hexanoic acid, cetyl 2-ethyl hexanoic acid, isopropyl myristic acid, butyl myristic acid, isopropyl palmitic acid, ethyl stearic acid, octyl palmitic acid, isocetyl isostearic acid, butyl stearic acid, ethyl linoleic acid, isopropyl linoleic acid, ethyl oleic acid, isocetyl myristic acid, isostearyl myristic acid, isostearyl palmitic acid, octyldodecyl myristic acid, isocetyl isostearic acid, diethyl sebacic acid, isopropyl adipic acid, isoalkyl neopetanoic acid, glyceryl tri(capryl, capric acid), trimethylopropane tri-2-ethyl hexanoic acid, trimethylopropane triisostearic acid, pentaerythritol tetra-2 ethyl hexanoic acid, cetyl caprylic acid, decyl lauric acid, hexyl lauric acid, decyl myristic acid, myristyl myristic acid, cetyl myristic acid, stearyl stearic acid, decyl oleic acid, cetyl licinoleic acid, isostearyl lauric acid, isotridecyl myristic acid, isocetyl palmitic acid, octyl stearic acid, isocetyl stearic acid, isodecyl oleic acid, octyldodecyl oleic acid, octyldodecyl linoleic acid, isopropyl isostearic acid, cetostearyl 2-ethyl hexanoic acid, stearyl 2-ethyl hexanoic acid, hexyl isostearic acid, ethylene glycol dioctanoic acid. ethylene glycol dioleic acid, propylene glycol dicapric acid, propylene glycol di(capryl, capric acid), propylene glycol dicaprylic acid, neopentylglycol dicapric acid, neopentylglycol dioctanoic acid, glyceryl tricaprylic acid, glyceryl triundecylic acid, glyceryl triisopalmitic acid, glyceryl triisostearic acid, octyldodecyl neopentanoic acid, isostearyl octanoic acid, octyl isononanoic acid, hexyldecyl neodecanoic acid, octyldodecyl neodecanoic acid, isocetyl isostearic acid, isostearyl isostearic acid, octyldecyl isostearic acid, polyglycerin oleanoic acid ester, polyglycerin isostearic acid ester, triisocetyl citric acid, triisoalkyl citric acid, triisooctyl citric acid, lauryl lactic acid, myristyl lactic acid, cetyl lactic acid, octyldecyl lactic acid, triethyl citric acid, acetyltriethyl citric acid, acetyl tributyl citric acid, trioctyl citric acid, diisostearyl maleic acid, di 2-ethylhexyl hydroxy stearic acid, 2-ethyl hexyl succinic acid, diisobutyl adipic acid, diisopropyl sebasinic acid, dioctylsebacinic acid, cholesteryl stearic acid, cholesteryl isostearic acid, cholesteryl hydroxy stearic acid, cholesteryl hydroxy stearic acid, cholesteryl oleic acid, dihydrocholesteryl oleic acid, pitsteryl isostearic acid, pitsteryl oleic acid, isocetyl 12-stealoyl hydroxy stearic acid, stearyl 12-stealoyl hydroxy stearic acid, isostearyl 12-stealoyl hydroxy stearic acid. Preferable hydrocarbon oil described above may comprise squalene, liquid paraffin, α-olefin oligomer, isoparaffin, ceresin, paraffin, liquid isoparaffin, polybuden, microcrystalline wax, vaselin and the like.

Preferable silicone oil may comprisepolymethylsilicone, methylphenylsilicone, methylcyclopolysiloxane, octamethylpolysiloxane, decamethylpolysiloxane, dodecamethylcyclosiloxane, dimethyl siloxane-methyl cetyloxysiloxan copolymer, dimethyl siloxane-methyl stealoxysiloxane copolymer, alkyl modified silicone oil, amino modified silicone oil and the like.

Preferable fluoride oil can comprise perfluoropolyether and the like. Preferable animal or plant oil can comprise avocado oil, almond oil, olive oil, sesame oil, rice husk oil, safflower oil, soy-bean oil, corn oil, rape oil, amygdalin oil, palm kernel oil, palm oil, pimaja oil, sunflower oil, fruit seed oil, cotton seed oil, coconut palm oil cucui nut oil, wheat embryo bud oil, rice embryo bud oil, sia butter, evening-primrose oil, marker daymia nut oil, medo home oil, egg yolk oil, lanolin, hempseed oil, mink oil, orange ruppy oil, hohoba oil, carnauba wax, liquid lanolin, solid pimaja wax and the like. Preferable humectants can comprise water-soluble low molecular humectants, lipophilic low molecular humectants, water-soluble polymer and lipid soluble polymer.

Specifically, preferable water soluble low molecular humectants can comprise cerin, glutamine, sorbitol, mannitol, pyrrolidone-carboxylic acid Na, glycerin, propylene glycol, 1,3-butylene glycol, ethylene glycol, polyethylene glycol (polymerization index. >2), polypropyleneglycol (polymerization index >2), lactic acid, lactate salt and the like.

Preferable lipid soluble low molecular humectants can comprise cholesterol, cholesteryl ester and the like.

Preferable water soluble polymer can comprise carboxy vinyl polymer, poly asparaginic acid salt, tragacanth, xanthin gum, HMC (hydroxy methyl celluose), HEC (hydroxy ethyl celluose), HPC (hydroxy propyl celluose), carboxymethylcellulose, water soluble chitin, chitosan, dextrin and the like.

Preferable lipid soluble polymer can comprise polyvinylpyrrolidone-eicocene copolymer, polyvinylpyrrolidone-hexadecene copolymer, nitrocellulose, dextrin fatty acid ester, silicone polymer and the like.

Preferable emollients can comprise long chain acyl glutamic acid cholesteryl ester, cholesteryl hydroxy stearic acid, 12-hydroxy stearic acid, rogic acid, lanolin fatty acid cholesteryl ester and the like.

Preferable surfactant can comprise nonionic surfactants, anionic surfactants, cationic surfactants, ambivalent surfactants and the like.

Specifically, preferable non-ionic surfactants can comprise self-emulsified monostearic acid glycerin, propylene glycol fatty acid ester, glycerin fatty acid ester, polyglycerin fatty acid ester, sorbitan fatty acid ester, polyoxyethylene (POE) sorbitan fatty acid ester, POE sorbitan fatty acid ester, POE glycerin fatty acid ester, POE alkyl ether, POE fatty acid ester, POE solid pimaja oil, POE pimaja oil, POE-POP copolymer, POE-POP alkyl ether, polyether modified silicone, lauric acid alkanol amide, alkyl amine oxide, hydrogen addition soybean phospholipid and the like.

Preferable anionic surfactants can comprise fatty acid soap, -acyl sulfonic acid salt, alkyl sulfonic acid salt, alkyl ally sulfonic acid, alkyl naphthalene sulfonic acid salt, alkyl sulfonic acid salt, POE alkylether sulfate salt, alkyl amide sulfate salt, alkyl phosphate salt, POE alkyl phosphate salt, alkylamide phosphate salt, alkyloylalkyl taurine salt, N-acyl-amino acid salt, POE alkyl ether carboxylic acid salt, alkyl sulfo succinic aid salt, alkyl sulfo-acetic acid salt, acylated hydrolysable collagen peptide salt, perfluoro alkyl phosphate ester and the like.

Preferable cationic surfactant can comprise alkyl trimethyl ammonium chloride, stearyl trimethyl ammonium chloride, stearyl trimethyl ammonium bromide, setostearyltrimethyl ammonium chloride, distearyl dimethyl ammonium chloride, stearyl dimethyl benzyl ammonium chloride, vehenyltrimethyl ammonium bromide, benzalkonium chloride, diethylamino ethyl amide stearic acid, dimethylaminopropyl amide stearic acid, lanolin derivatives quaternary ammonium and the like.

Preferable ambivalent surfactants can comprise carboxy betaine type, amide betaine type, hydroxy sulfo betaine type, phosphobetaine type, aminocarboxylic acid, imidazoline derivatives type, amide amine type and the like.

Preferable organic and inorganic dyes can comprise silicic acid, anhydrous silicic acid, magnesium silicic acid, talc, ceracyte, mica, caolin, bengala, clay, bentonite, titan film mica, oxy chlorine bismuth, zirconium oxide, magnesium oxide, zinc oxide, titan oxide, aluminium oxide, calcium sulfate, barium sulfate, magnesium sulfate, calcium carbonate, magnesium carbonate, ferrous oxide, chromium oxide, chromium hydroxide, calamine, carbon black and the complex thereof as an inorganic dyes; polyamide, polyester, polypropylene, polystyrene, polyurethane, vinyl resin, urea resin, phenol resin, fluoride resin, silicone resin, acryl resin, melamine resin, epoxy resin, polycarbonated resin, divinyl benzene-styrene copolymer, silk powder, cellulose, CI pigment yellow, CI pigment orange as an organic dyes; and their complex etc.

Preferable organic powder can comprise metal soap such as calcium stearate; alkyl phosphonate metal salt such as sodium zinc cetylic acid, zinc laurylic acid, calcium laurylic acid; acylamino acid polyvalent metal salt such as calcium N-lauroyl-alanine, zinc N-lauroyl-alanine, calcium N-lauroyl-glycine etc.; amide sulfonic acid polyvalent metal salt such as calcium N-lauroyl-taurine, calcium N-palmitoyl-taurine; N-acyl basic amino acid such as Nc-lauroyl-L-lysine, Nc-palmitoyl-lysine, N-palmitoyl ornitine, N-lauroly arginine, hardened lanolin fatty acid acyl arginine and the like; N-acylpolypeptide such as N-lauroylglycyl glycine; -amino fatty acid such as amino caprylic acid, amino lauric acid and the like; polyethylene, polypropylene, nylon, polymethylmetacrylate, polystyrene, divinylbenzene-styrene copolymer, ethylene tetrafluoride and so on.

Preferable ultraviolet absorbing agents can comprise paraaminobenzoic acid, paraamonoethyl benzoate, paraamino amyl benzoate, paraamino octyl benzoate, ethyleneglycol salicylate, phenyl salicylate, octyl salicylate, benzyl salicylate, butylphenyl salicylate, homomentyl salicylate, benzyl cinnamic acid, paramethoxy 2-ethoxy ethyl cinnamic acid, paramethoxy octyl cinnamic acid, diparamethoxy mono-2-ethylhexane glyceryl cinnamic acid, paramethoxy isopropyl cinnamic acid, diisopropyl-diisopropyl cinnamate ester mixture, urokanic acid, ethyl urokanic acid, hydroxy methoxy benzophenone, hydroxymethoxy benzophenone sulfonic acid and their salt, dihydroxy methoxy benzophenone, dihydroxy methoxy benzophenone disulfonate Na, dihydroxy benzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl) benzotriazole and the like.

Preferable preservatives can comprise hinokitiol, trichloric acid, trichlorohydroxydiphenylether, chlorohexidine glucuronate, phenoxyethanol, resorcine, isopropylmethylphenol, azulene, salicylic acid, zinc pilithione, bezalconium HCl, photosensitizer 301, mononitroguaiacol Na, undecylenic acid etc.

Preferable antioxidants can comprise butylhydroxyanisole, propyl gallate, ellisorbate and the like.

Preferable pH controller can comprise citric acid, sodium citrate, malic acid, sodium malate, fumaric acid, sodium fumaric acid, succinic acid, sodium succinic acid, sodium hydroxide, sodium hydrogen phosphate and the like.

Preferable alcohol can comprise cetyl alcohol etc.

Furthermore, other ingredient addable to above described component and the amount thereof is not limited within the scope of the purpose and effect of the present invention, however, it is preferable that the amount of the other ingredients ranges from 0.01 to 10%, more preferably, 0.01 to 5% in that of total composition.

The cosmetic composition of the present invention can be modified as a solution, emulsion, cohesive mixture etc.

Above described ingredients such as water-soluble vitamin, lipid soluble vitamin, peptide polymer, polysaccharide polymer, sphingolipid, sea weed extract and addable ingredients which can be added other than above described ingredients if necessary, can be obtained by conventional methods disclosed in the literature (Matsumoto Mithio; Manual for the development of transdermal applied preparation. Seisi Press, 1^{st}Ed., 1985).

Specifically, cosmetic formulation of the present invention may be prepared in any form well-known in the art for example, skin lotion, skin softener, skin toner, astringent, lotion, milk lotion, moisture lotion, nutrient lotion, massage lotion, nutrient cream, moisture cream, hand cream, foundation, essence, nutrient essence, pack, soap, cleansing foam, cleansing lotion, cleansing cream, body solution, body cleanser and the like.

More specifically, the paste, cream or gel formulation of the present invention may use lactose, talc, silica, aluminum hydroxide, calcium silicate or polyamide powder etc. as a cosmetic carrier and further add chlorofluorohydrocarbon, propane/butane or dimethyl ether as an expellant.

More specifically, the solution of emulsion formulation of the present invention may use solvent, solubilizer, or emulsifier such as water, ethanol, isopropanol, ethyl carbonate, ethyl acetate, benzyl alcohol, benzyl benzoate, propylene glycol, 1,3-butylene glycol, glycerol fatty ester, PEG or sorbitan fatty ester etc.

More specifically, the suspension formulation of the present invention may use appropriate carrier, for example, liquid dilution such as water, ethanol or PEG emulsifier such as ethoxylated isostearyl alcohol, polyoxyethylene sorbitol ester, or polyoxyethylene sorbitane ester; and microcrystalline cellulose, aluminum meta hydroxide, bentonite, agar or traganth etc.

More specifically, the surfactant comprising cleansing formulation of the present invention may use fatty alcohol sulfate, fatty alcohol ether sulfate, sulfosuccinic acid monoester, icetionate, imidazolinum derivative, methyl taurate, sarocynate, fatty acid amide ethyl sulfate, alkyl amido betaine, fatty alcohol, fatty acid glyceride, fatty acid diethanol amide, plant oil, linolic acid, or ethoxylated glycerol fatty acid ester etc.

Inventive compounds of the present invention have no toxicity and adverse effect therefore can be used with safe.

### Advantageous Effects

The present invention relates to a composition comprising an extract of combined herbs consisting of Acanthopanax koreanum Nakai and Crinum asiaticum var. japonicum showing preventing activity of baldness disorder and stimulating activity of hair growth.

The inventive combined extract showed potent hair-growth promoting activity and synergistic effect than each herb, i.e., the sole treatment of Acanthopanax koreanum Nakai and Crinum asiaticum var. japonicum, through the animal model experiments such as the growth rate test using by C57BL/6 mouse, to confirm the effect the inventive combined extract, i.e., combined treatment of Acanthopanax koreanum Nakai and Crinum asiaticum var. japonicum.

### DESCRIPTION OF DRAWINGS

### Best Mode

The above and other objects, features and other advantages of the present invention will more clearly understood from the following detailed description taken in conjunction with the accompanying drawings, in which;

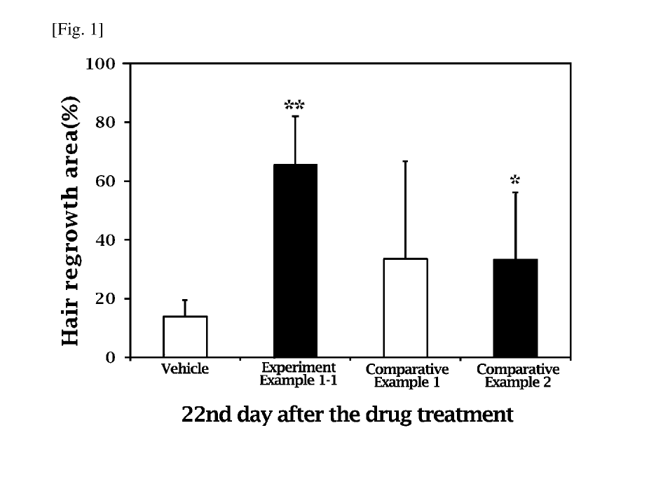

FIG. 1 shows the hair-growth promoting activity of the comparative test groups (AE & CE) and test sample group (ACE1);

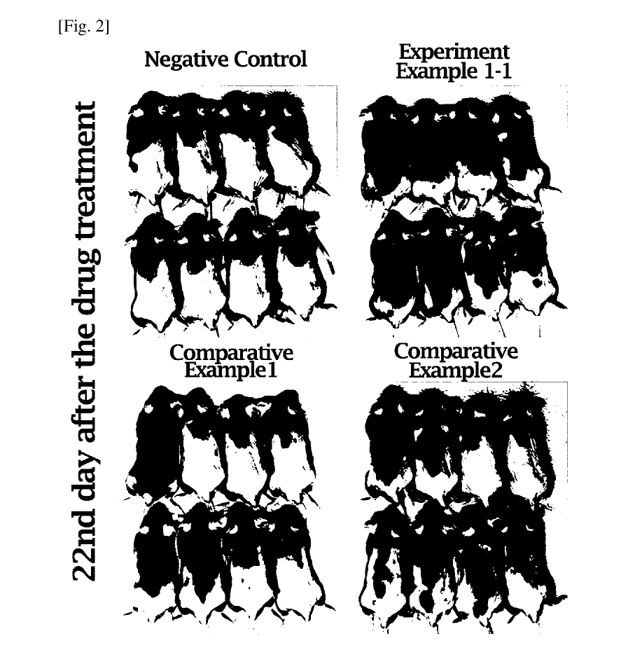

FIG. 2 shows the hair-growth promoting activity of the comparative test groups (AE & CE) and test sample group (ACE1).

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Comparative Example 1

### Preparation of the Extract of Acanthopanax koreanum Nakai

500 g of Acanthopanax koreanum Nakai (Cheonan) was washed with tap water, dried for one day, cut and mixed with 8 fold weight of 50% (v/v) ethanol. The solution was extracted for 2 hours with reflux extraction at above 90° C. and the resulting residue was filtered with a filter paper (Whatman Co.). The filtrate was concentrated with rotary evaporator (Heidolph, Laboota 4001) and dried with freeze dryer (FDU-210, EYELA) to obtain powdered extract of Acanthopanax koreanum Nakai (designated as “AE” hereinafter). The powder was used as a comparative sample in the following

### Experimental Examples

### Comparative Example 2

### Preparation of the Extract of Crinum asiaticum Var. japonicum

500 g of Crinum asiaticum var. japonicum (Cheju-do) was washed with tap water, dried for one day, cut and mixed with 10 fold weight of 50% (v/v) ethanol. The solution was extracted for 2 hours with reflux extraction at above 90° C. and the resulting residue was filtered with a filter paper (Whatman Co.). The filtrate was concentrated with rotary evaporator (Heidolph, Laboota 4001) and dried with freeze dryer (FDU-210, EYELA) to obtain powdered extract of Crinum asiaticum var. japonicum (designated as “CE” hereinafter). The powder was used as a comparative sample in the following Experimental Examples.

### Example 1

### The Combination of Dried Extract of Each Herb

### 1-1. ACE1 Combination

The extract of Acanthopanax koreanum Nakai and Crinum asiaticum var. japonicum prepared in Comparative Examples, was mixed with the ratio of 1:1 (w/w) (designated as “ACE1” hereinafter). The powder was used as a test sample in the following Experimental Examples.

### 1-2. ACE2 Combination

The extract of Acanthopanax koreanum Nakai and Crinum asiaticum var. japonicum prepared in Comparative Examples, was mixed with the ratio of 2:1 (w/w) (designated as “ACE2” hereinafter). The powder was used as a test sample in the following Experimental Examples.

### 1-3. ACE3 Combination

The extract of Acanthopanax koreanum Nakai and Crinum asiaticum var. japonicum prepared in Comparative Examples, was mixed with the ratio of 1:2 (w/w) (designated as “ACE3” hereinafter). The powder was used as a test sample in the following Experimental Examples.

### 1-4. ACE4 Combination

The extract of Acanthopanax koreanum Nakai and Crinum asiaticum var. japonicum prepared in Comparative Examples, was mixed with the ratio of 3:1 (w/w) (designated as “ACE4” hereinafter). The powder was used as a test sample in the following Experimental Examples.

### 1-5. ACE5 Combination

The extract of Acanthopanax koreanum Nakai and Crinum asiaticum var. japonicum prepared in Comparative Examples, was mixed with the ratio of 1:3 (w/w) (designated as “ACE5” hereinafter). The powder was used as a test sample in the following Experimental Examples.

### Example 2

### The Combination of Extract of Combined Herbs

### 2-1. ACC1 Combination

Acanthopanax koreanum Nakai (Cheonan) and Crinum asiaticum var. japonicum (Cheju-do) were washed with tap water, dried, cut and mixed with the ratio of 1:1 (w/w) to obtain 300 g of the dried mixture. The mixture was added to 8 fold volume of 50% (v/v) ethanol. The solution was extracted for 2 hours with reflux extraction at above 90° C. and the resulting residue was filtered with a filter paper (Whatman Co.). The filtrate was concentrated with rotary evaporator (Heidolph, Laboota 4001) and dried with freeze dryer (FDU-210, EYELA) to obtain the extract of combined herbs (designated as “ACC1” hereinafter). The powder was used as a comparative sample in the following Experimental Examples.

### 2-2. ACC2 Combination

The Acanthopanax koreanum Nakai and Crinum asiaticum var. japonicum was mixed with the ratio of 2:1 (w/w) to obtain 300 g of the dried mixture. The mixture was extracted and the filtrate was concentrated and dried according to the similar method to those disclosed in Example 2-1 to obtain the extract of combined herbs (designated as “ACC2” hereinafter). The powder was used as a test sample in the following Experimental Examples.

### 2-3. ACC3 Combination

The Acanthopanax koreanum Nakai and Crinum asiaticum var. japonicum was mixed with the ratio of 3:1 (w/w) to obtain 300 g of the dried mixture. The mixture was extracted and the filtrate was concentrated and dried according to the similar method to those disclosed in Example 2-1 to obtain the extract of combined herbs (designated as “ACC3” hereinafter). The powder was used as a test sample in the following Experimental Examples.

### Experimental Example 1

### Promoting Effect on Hair Growth

To confirm the effect of inventive extract on the preventing activity of baldness disorder and stimulating activity of hair growth, the following method was performed according to the method disclosed in the literature (Hee Kyoung Kang et al., Eur. J. Dermatol., 20(1), pp42-48, 2010).

### 1-1. Preparation

5 weeks-old mouse (C57BL/6, 15-18 g, at the beginning of catagen, Central Labs. Animal Inc. Seoul, Korea) was accustomed to the breeding environment for 1 week and anesthetized intramuscularly with an anesthetizer (2.5 mg/mouse, Ketamine, rompun, Bayer Korea Inc.). The back-hair of anesthetized mouse was removed by a grainer and the mice were divided into three groups, i.e., (1) negative control group: treatment group with only adjuvant, (2) comparative test group: treatment group with the extract prepared in Comparative Examples dissolved in adjuvant in a dose dependent manner, (3) test sample group: treatment group with the extract prepared in Examples dissolved in adjuvant in a dose dependent manner. 0.2 ml of the test samples was spread on the back of mouse once a day for 4 weeks.

### 1-2. Hair-Growth Promoting Effect (Example 1-1)

The ratio of hair-regrown area/shaved area was quantitatively analyzed by image analyzer (Image-Pro, USA) after shooting the hair-grown area with digital camera (Canon, Japan) and the result was shown in Tables 1 to 4 & FIGS. 1 to 2.

TABLE 1

| | | | | ratio of hair-regrown |
|---|---|---|---|---|
| | | | | area/shaved area (%) |
| | | Concentration | No. of | 22nd day after |
| Group | | (mg/0.2 ml) | animal | the treatment |
| negative control | | 0 | 8 | 13.9 ± 5.6 |
| group | | | | |
| Example 1-1 | | 1.0 + 1.0 = 2.0 | 8 | 65.5 ± 16.4** |
| (ACE1) | | | | |
| comparative | 1(AE) | 2.0 | 8 | 33.6 ± 33.1 |
| test group | 2(CE) | 2.0 | 8 | 33.4 ± 22.8* |

(Mean ± S.D.): **p < 0.05 -> 0.01, *p < 0.05

At the result, the dorsal skin after removing hair showed pink color since the mice were at the stage of catagen and the ratio of hair-regrown area/shaved area in the negative control group showed 13.9% at 22nd day after the treatment. The ratio of hair-regrown area/shaved area in the test sample group treated with the extract (Example 1-1) showed 65.5% at 22nd day while those in the comparative test group 1 and 2 showed only 33.6% and 33.4%, respectively, which result confirmed that the inventive combined extract, i.e., combined treatment of Acanthopanax koreanum Nakai and Crinum asiaticum var. japonicum, showed potent hair-growth promoting activity and synergistic effect than each herb, i.e., the sole treatment of Acanthopanax koreanum Nakai or Crinum asiaticum var. japonicum.

### 1-3. Hair-Growth Promoting Effect (Example 1-2 & 1-3)

After spreading the test samples on the back of mice for 25 days, the hair-growth promoting effect was photographed.

TABLE 2

| | | | ratio of hair-regrown |
|---|---|---|---|
| | Concentration | No. of | area/shaved area (%) |
| Group | (mg/0.2 ml) | animal | 25th day after the treatment |
| negative | 0 | 8 | 4.6 ± 5.0 |
| control group | | | |
| Example 1-2 | 1.3 + 2.7 = 4.0 | 8 | 46.5 ± 24.4** |
| (ACE2) | | | |
| Example 1-3 | 2.7 + 1.3 = 4.0 | 8 | 65.5 ± 32.6** |
| (ACE3) | | | |

(Mean ± S.D.): **p < 0.01

At the result, the dorsal skin after removing hair showed pink color since the mice were at the stage of catagen and the ratio of hair-regrown area/shaved area in the negative control group showed 4.6% at 25th day after the treatment. The ratio of hair-regrown area/shaved area in the test sample group treated with the extract disclosed in Example 1-2 & Example 1-3, showed 46.5% and 65.5%, respectively, at 25th day, which result confirmed that the inventive combined extract, i.e., combined treatment of Acanthopanax koreanum Nakai and Crinum asiaticum var. japonicum, showed potent hair-growth promoting activity.

### 1-4. Hair-Growth Promoting Effect (Example 1-4 & 1-5)

After spreading the test samples on the back of mice for 21 days, the hair-growth promoting effect was photographed.

TABLE 3

| | | | ratio of hair-regrown |
|---|---|---|---|
| | Concentration | No. of | area/shaved area (%) |
| Group | (mg/0.2 ml) | animal | 21st day after the treatment |
| negative | 0 | 8 | 24.8 ± 29.1 |
| control | | | |
| group | | | |
| Example 1-4 | 0.5 + 1.5 = 2.0 | 8 | 67.0 ± 26.8** |
| (ACE4) | | | |
| Example 1-5 | 1.5 + 0.5 = 2.0 | 8 | 70.4 ± 33.3** -> 70.4 ± 30.3** |
| (ACE5) | | | |

(Mean ± S.D.): **p < 0.01

At the result, the dorsal skin after removing hair showed pink color since the mice were at the stage of catagen and the ratio of hair-regrown area/shaved area in the negative control group showed 24.8% at 21st day after the treatment. The ratio of hair-regrown area/shaved area in the test sample group treated with the extract disclosed in Example 1-4 & Example 1-5, showed 67.0% and 70.4%, respectively, at 21st day, which result confirmed that the inventive combined extract, i.e., combined treatment of Acanthopanax koreanum Nakai and Crinum asiaticum var. japonicum, showed potent hair-growth promoting activity.

### 1-5. Hair-Growth Promoting Effect (Example 2-1 & 2-2)

After spreading the test samples on the back of mice for 22 days, the hair-growth promoting effect was photographed.

TABLE 4

| | | | ratio of hair-regrown |
|---|---|---|---|
| | Concentration | No. of | area/shaved area (%) |
| Group | (mg/0.2 ml) | animal | 22nd day after the treatment |
| negative | 0 | 8 | 23.4 ± 19.0 |
| control group | | | |
| Example 2-1 | 4.0 + 4.0 = 8.0 | 8 | 66.3 ± 32.4* |
| (ACC1) | | | |
| Example 2-2 | 2.7 + 5.3 = 8.0 | 8 | 46.3 ± 34.3 |
| (ACC2) | | | |

(Mean ± S.D.): *p < 0.05

At the result, the dorsal skin after removing hair showed pink color since the mice were at the stage of catagen and the ratio of hair-regrown area/shaved area in the negative control group showed 23.4% at 22nd day after the treatment. The ratio of hair-regrown area/shaved area in the test sample group treated with the extract disclosed in Example 2-1 & Example 2-2, showed 66.3% and 46.3%, respectively, at 21st day, which result confirmed that the inventive combined extract, i.e., combined treatment of Acanthopanax koreanum Nakai and Crinum asiaticum var. japonicum, showed potent hair-growth promoting activity.

Hereinafter, the formulating methods and kinds of excipients comprising the inventive extract of the present invention, will be described, but the present invention is not limited to them. The representative preparation examples are described as follows.

| | |
|---|---|
| Extract (ACE1) | 0.5% (w/w) |
| while vaseline | 2.5% (w/w) |
| stearyl alcohol | 0.22% (w/w) |
| ethyl (or methyl) p-oxybenzoate | 0.25% (w/w) |
| propylene glycol | 12.0% (w/w) |
| sodium lauryl sulfate | 0.15% (w/w) |
| propyl p-oxybenzoate | 0.15% (w/w) |
| Distilled water | up to 100% |

Ointment preparation was prepared by mixing the above components, filling in a ointment tube by conventional ointment preparation method.

### Preparation of Hair Tonic

| | |
|---|---|
| Extract (ACE2) | 10.0% |
| Menthol | 0.05% |
| Panthenol | 0.2% |
| salicylic acid | 0.1% |
| tocopherol acetate | 0.1% |
| salicylic acid | 0.1% |
| pyridoxine•HCl | 0.1% |
| castor oil | 5.0% |
| pigment | optimum amount |
| Flavor | optimum amount |
| Ethanol | optimum amount |
| Distilled water | up to 100% |

Hair tonic preparation was prepared by dissolving the active components according to conventional hair tonic preparation method.

### Preparation of Skin Conditioner

| | |
|---|---|
| Extract (ACE3) | 2.5% |
| Cetanol | 3.5% |
| self-emulsifying mono-stearate glycerol | 1.5% |
| Propylene glycol | 2.5% |
| stearyl methyl benzyl ammonium chloride (25%) | 7.0% |
| methyl p-oxybenzoate | 0.3% (w/w) |
| pigment | optimum amount |
| Flavor | optimum amount |
| Distilled water | up to 100% |

Skin conditioner preparation was prepared by dissolving the active components according to conventional skin conditioner preparation method.

### Preparation of Hair Lotion

| | |
|---|---|
| Extract (ACE4) | 5.0% |
| Resorcinol | 2.0% |
| Menthol | 2.0% |
| Panthenol | 0.5% |
| piroctone olamine | 0.1% |
| Flavor & pigment | 0.5% |
| Distilled water | up to 100% |

Hair lotion preparation was prepared by dissolving the active components according to conventional hair lotion preparation method.

### Preparation of Hair Soap

| | |
|---|---|
| Extract (ACC1) | 0.1% |
| titanium dioxide | 0.2% |
| polyethylene glycol | 0.8% |
| glycerin | 0.5% |
| ethylene diamine tetracetate | 0.05% |
| sodium | 1.0% |
| pigment | optimum amount |
| soap flavor | optimum amount |
| beauty soap base (humidity 13%) | up to 100% |

Hair soap was prepared by dissolving the active components according to conventional hair soap preparation method.

### Preparation of Cream

| | |
|---|---|
| Extract (ACC2) | 3.0% |
| Polyethyleneglycomonosterate | 2.0% |
| Monostearate glycerin | 1.0% |
| Cetyl alcohol | 4.0% |
| Squalene | 6.0% |
| Tri 2-glycerly ethylhexanoate | 6.0% |
| Sphingo-glycolipid | 1.0% |
| 1,3-butylene glycol | 3.0% |
| Distilled water | up to 100% |

Cream preparation was prepared by dissolving the active components according to conventional cream preparation method.

### Preparation of Beauty Solution

| | |
|---|---|
| Extract (ACC3) | 2.0% |
| Hydroxyethylene cellulose (2% solution) | 12.0% |
| Xanthin gum (2% solution) | 2.0% |
| 1,3-butylene glycol | 3.0% |
| Glycerin concentration | 4.0% |
| Sodium hyaluronte | 5.0% |
| Distilled water | 100 ml |

Beauty solution preparation was prepared by dissolving the active components according to conventional beauty solution preparation method

The invention being thus described as will be obvious that it may be varied in many ways. Such variations are not to be regarded as a departure from the spirit and scope of the present invention, and all such modifications as would be obvious to those skilled in art are intended to be included within the scope of the following claims.

### INDUSTRIAL APPLICABILITY

As described in the present invention, The present invention relates to a composition comprising an extract of combined herbs consisting of Acanthopanax koreanum Nakai and Crinum asiaticum var. japonicum showing preventing activity of baldness disorder and stimulating activity of hair growth.

The inventive combined extract showed potent hair-growth promoting activity and synergistic effect than each herb, i.e., the sole treatment of Acanthopanax koreanum Nakai or Crinum asiaticum var. japonicum, through the animal model experiments such as the growth rate test using by C57BL/6 mouse, to confirm the effect the inventive combined extract, i.e., combined treatment of Acanthopanax koreanum Nakai and Crinum asiaticum var. japonicum.

1. An external pharmaceutical composition comprising an extract of combined herbs consisting of Acanthopanax koreanum Nakai and Crinum asiaticum var. japonicum as active ingredients for treating a hair baldness disorder and stimulating activity of hair growth.

2. The external pharmaceutical composition according to claim 1, wherein the extract is extracted with a solvent selected from the group of consisting water, spirits, C₁-C₄ lower alcohol and mixtures thereof.

3. The external pharmaceutical composition according to claim 1, wherein the extract is selected from the group consisting of an extract from a combination of Acanthopanax koreanum Nakai and Crinum asiaticum var. japonicum with mixed ratio of 1-10:10-1 (w/w), and a combination of an extract from Acanthopanax koreanum Nakai and an extract from Crinum asiaticum var. japonicum with mixed ratio of 1-10:10-1 (w/w).

4. The external pharmaceutical composition according to claim 1, wherein the hair baldness disorder is selected from the group consisting of androgentic alopecia, alopecia seniles, and alopecia areata.

5. The external pharmaceutical composition according to claim 1, wherein the composition is in a form selected from the group consisting of cream, gel, patch, ointment, spray solution, liniment, cataplasma, lotion, gel, balm, paste and aerosol.

6. A cosmetic composition comprising an extract of a combination of herbs consisting of Acanthopanax koreanum Nakai and Crinum asiaticum var. japonicum as active ingredients for improving a hair baldness disorder and stimulating activity of hair growth.

7. The cosmetic composition according to claim 6, wherein the composition is in a form selected from the group consisting of hair tonic, hair conditioner, hair essence, hair lotion, hair nutrient lotion, hair shampoo, hair rinse, hair treatment, hair cream, hair nutrient cream, hair was, hair moisture cream, hair massage cream, hair aerosol, hair pack, hair nutrient pack, hair soap, hair cleaning foam, pomade, hair drying agent, hair care agent, hair dyeing agent, hair permanent wave agent, hair bleaching agent, hair gel, hair glaze, hair dressing pomade, hair lacquer, hair moisturizer, hair mousse, eyebrows nutrient, eyelash nutrient, hair spray, astringent, nutrient lotion, nutrient cream, massage cream, essence, pack, foundation, cleansing water, soap, treatment, beauty solution and combinations thereof.

8. (canceled)

9. A method of treating a baldness disorder in a mammal or human in need thereof comprising the step of administering to the mammal or human with a therapeutically effective amount of an extract from a combination of Acanthopanax koreanum Nakai and Crinum asiaticum var. japonicum, together with a pharmaceutically acceptable carrier thereof.
